(19) United States
(12) Patent Application Publication
(10) Pub. No.: US 2011/0283770 A1
(43) Pub. Date: Nov. 24, 2011

(54) BREATH ANALYSIS

(75) Inventor: Bertil Hok, Vasteras (SE)

(73) Assignee: HOK INSTRUMENT AB, Vasteras (SE)

(21) Appl. No.: 13/147,496

(22) PCT Filed: Feb. 8, 2010
(86) PCT No.: PCT/SE2010/050146

§ 371 Date: Aug. 2, 2011

(30) Foreign Application Priority Data
Feb. 10, 2009 (SE) 0900162-9

(51) Int. Cl. G01N 33/497

(52) U.S. Cl. 73/23.3

(57) ABSTRACT

A method and apparatus for breath analysis from a test person who may be incapable of delivering controlled forced expiration. Breath sampling may be essentially contactless and is provided by active air intake to measuring cell (4) via receptor (2) which exhibits concentration preserving effect based on partial reflow which prevents mixing with ambient air. The analysis is performed by IR spectroscopy, including the determination of concentration of a volatile organic substance, e g ethyl alcohol, and a physiological reference substance, e g carbon dioxide. The timing of sampling is controlled by starting command from operator, and the duration of air intake is controlled by sensor signal from the measuring cell. The apparatus includes one hand unit (1), an exchangeable receptor (2), and a docking station (3).

The present invention is concerned with broadening the present applicability of breath analysis for the detection of volatile substances, e g ethyl alcohol in expired breath. The present applicability has serious limitations with respect to the degree of cooperation, and the physiological capabilities required by the test person. The present invention enables breath analysis from incapable persons, by which is meant persons unable to cooperate by providing forced expiration, and persons with a physical or psychological handicap with decreased lung capacity.

At present, determination of the alcohol concentration of an unconscious patient within the health care system can only be performed by laboratory analysis of blood samples, which is costly and often takes more than one hour. Breath analysis is not practical in unconscious patients, since alcometers according to the state of the art require active cooperation from the test person, including forced expiration of a sample volume of 0.7-1.2 liters, using a tightly fitting mouthpiece.

Swedish emergency care receives approximately 50 000 cases of unconscious patients annually, mainly patients with stroke, epilepsy, cardiac infarct, coma, trauma or intoxication from alcohol or drugs. According to public statistics, approximately 250 persons die each year in Sweden as a consequence of acute alcohol intoxication, and this is also the most common cause of intoxication.

The absence of methods and equipment for objective and rapid determination of alcohol influence may lead to erroneous treatment or none at all when symptoms are difficult to interpret—in the worst case this may be a question of life or death. Access to rapid alcohol determination within emergency health care would reduce the risk that cardiac infarct, stroke, coma, or trauma, are mistaken for more easily handled alcohol intoxication.

Ambulance operators, rescue teams, clinicians at emergency care centers, and others would benefit from improved method and apparatus for breath analysis. Applications include sobriety tests performed by the police force, and alcolocks for the prevention of drunk driving. In these applications, it is generally required to deliver a vital capacity forced expiration, which is not only time consuming, but also requires a great deal of effort from the test person. Furthermore, the breath analyzer according to the state of the art is generally equipped with a replaceable mouthpiece for hygienic reasons. The test person is assumed to be capable of providing an airtight seal to the mouthpiece.

The prospective equipment and method for expanded applicability would have to meet the following requirements: Sampling and analysis should be basically independent of the test person's lung capacity, i e should require minimum sample volume and flow, and should be applicable both at spontaneous and assisted ventilation. An unconscious or handicapped person may exhibit significantly reduced tidal volume, i e the volume of each breath, compared to the normal value 500 ml of a passive breath. Furthermore, the demands are high with respect to easy operation, speed, accuracy, selectivity (identification of actual substances) reliability, protection against infection and environmental durability.

The present invention fulfills the requirements expressed above. Sampling can be performed, basically without physical contact by positioning a receptor designed for the purpose near the person's mouth and nose, however without sealing the opening, as is the case with mouthpieces according to prior art. The receptor according to the invention preferably exhibits a concentration preserving effect, without adding significant resistance to the respiratory airflow. The sample volume is less than 50 ml, i e a tenth of a normal passive breath. Determination of the concentration of ethyl alcohol or other volatile organic substance takes place directly in a measuring cell connected to the receptor. Air intake to the measuring cell is advantageously performed actively by means of a pump.

The concentration preserving effect of the receptor is based on its shape, including an inlet opening which, when sampling, is held in adjacent to the test person's mouth and nose. The area of the inlet opening is typically larger than the cross section of the mouth or nose openings of the test person. Thereby it will effectively collect the jet-like expiratory flow. The receptor is typically shaped like a cup, mug, funnel or scoop, with a partly confined inner volume which is preferably much smaller than the test person's tidal volume. The incident expiratory flow fills the inner volume, thereby evacuating the already present ambient air. The flow is distributed into one branch flowing into the measuring cell where the breath analysis takes place, and another branch which is redirected to the inlet opening. This reflowing branch forms a protective curtain, preventing the incident flow from mixing with ambient air, and will thus provide the effect of concentration preservation, i e the breath sample will not be diluted with ambient air.

By the concentration preserving effect, the substance concentration within the measuring cell will approach that of the inner airways, and the deviation may be considered insignificant compared with other error sources.

An important property of the present invention is the determination of a physiological reference substance, e g carbon dioxide, which occurs simultaneous with the alcohol determination. The concentration of the reference substance within the inner airways can be considered known, and therefore a measured value lower than normal is a sign that the sample has been diluted with ambient air. The determination of the reference substance in relation to the normal value is thus a quality measure of the analysis. The normal partial pressure of carbon dioxide is 4.8 kPa. Alternatively, water vapor can be used as physiological reference, with a normal value of 45 mg/l.

The simultaneous determination of a physiological reference means increased accuracy, minimization of the risk of false negative output, and simplified operation. The positioning of the receptor becomes less critical and the need for tight seal to the mouth or nose is eliminated. Further is obtained a univocal and permanent quality measure of the alcohol reading, or other unknown substance.

Before sampling, the measuring cell is automatically zeroed, followed by readiness indication. The analysis results are preferably presented within five seconds after finished sampling. Air flow through the measuring cell is only taking place at sampling and zeroing, resulting in full operating control of the measuring procedure. This is important for the reliability and environmental durability. The risk that the measuring cell will be exposed to destructive environmental influence is minimal.

Infrared (IR) spectroscopy is preferably used as measuring principle for both the unknown and the reference substances. The measuring cell is trans-illuminated by radiation from a broadband IR emitter hitting IR detectors with band pass filters for selected transmission bands. Ethyl alcohol is preferably detected as a decreasing transmittance in the wavelength range 3.3-3.5 μm compared to the reference level measured in clean air at zeroing. Carbon dioxide has a corresponding dip of transmission, or absorption peak, at 4.2-4.3 μm, and water at 2.5-2.8 μm.

In order to meet the requirements of accuracy the measuring cell should have a long transmission path for the IR radiation. The conflicting requirement of small overall physical size can be overcome by using an arrangement of multiple concave mirrors.

By IR spectroscopy it is possible both to identify and quantify all substances with absorption peaks in the actual wavelength range. IR spectroscopy has a fundamental advantage compared to other sensor principles spectroscopy with respect to reliability. On every measuring occasion it is possible to evaluate the stability of the baseline by comparing it to previous measurements. Thereby full control can be obtained of variations over time of the IR source, reflecting surfaces, filters, and detectors. Moreover, the sensitivity of the method is basically time independent, as opposed to sensors based on catalysis. The need for periodic calibration is therefore eliminated.

The detailed characteristics of the invention will be described in the following text relating to the drawings.

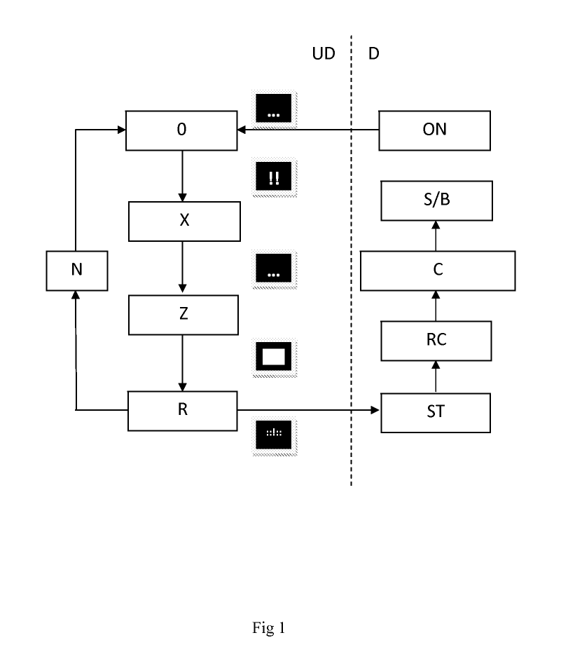

FIG. 1 shows a flow chart of the elements of an embodiment of the method according to the invention.

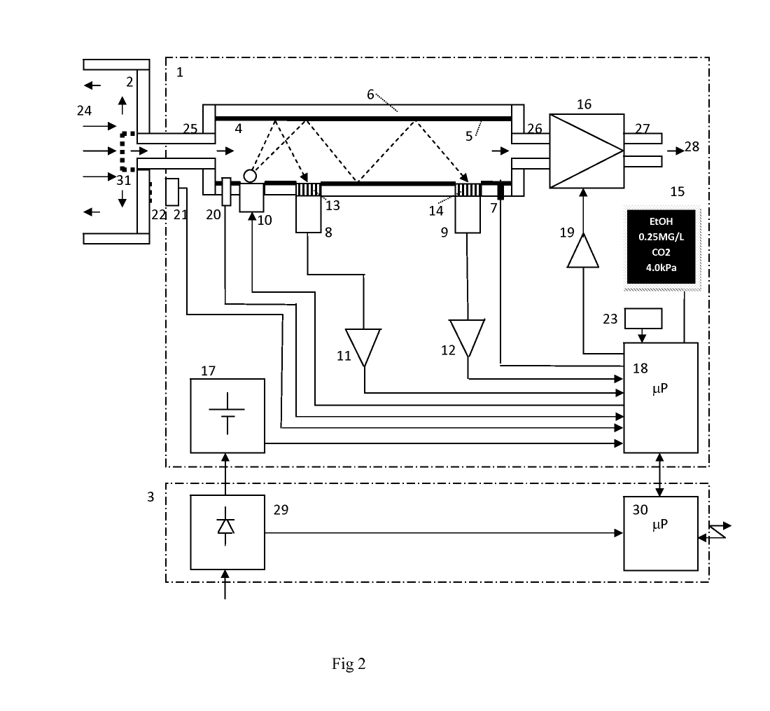

FIG. 2 shows a block diagram of a preferred embodiment of the apparatus according to the invention.

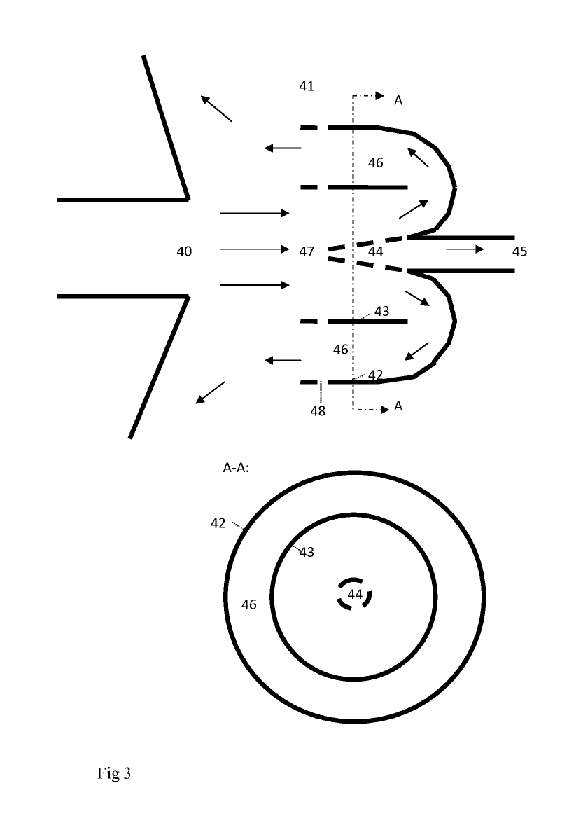

FIG. 3 shows schematically a preferred embodiment of the receptor according to the invention, and the relevant air flows.

The flow chart of FIG. 1 shows ten different states, each state being illustrated with a box. The ten states are divided into two categories, one marked “UD” referring to active states with respect to the actual breath analysis, whereas “D” are passive states between occasions of operation. The abbreviations refer to “undocked” and “docked”. The breath analyzer according to the invention includes a docking station for the handheld unit. In the active states, the handheld unit is undocked, and vice versa.

In a typical procedure according to the invention the apparatus is transferred from inactivated to active state category by moving the hand unit from the docking station, illustrated by the signature “ON”, Then automatic zeroing and function test of the measuring cell occurs at state “0”, while the display of the hand unit indicates a waiting condition, by the signature “. . . ” as illustrated in FIG. 1.

At zeroing ambient air is pumped into the measuring cell, and its zero level will therefore be related to ambient air at present air composition, which is basically zero both for organic substances and carbon dioxide. The function test is related to the absolute value of the measuring variables, in which the deviation from previous measurement, as well as baseline drift shall be within certain tolerances to be approved. Otherwise error indication will follow.

When zeroing has been performed, ready indication is provided by the signature “!”, and sampling “X” starts. The operator is then positioning the receptor and hand unit adjacent to the test person's mouth and nose and starts sampling by pressing a start button. Then air is pumped via the receptor through the measuring cell. Sampling and pumping continues until a certain CO₂ concentration is reached or longer. The operator may also choose to stop the sampling manually. Preferably, the sampling, analysis and signal presentation occurs in real time.

The analysis state “Z” may occur simultaneously or slightly after sampling, indicated by different signatures “. . . ” and “□”. The results of the analysis are thereafter presented on the display either graphically, numerically or in other form.

After result presentation the operator may choose to return the hand unit to the docking station or carry out a new measurement “N” whereby the procedure is repeated from zeroing and further. At docking a more extensive function test “ST” is performed, is performed together with charging of the battery of the hand unit “RC”. Finally the apparatus is transferred into a sleeping state, “S/B”.

FIG. 2 shows a block diagram of a preferred embodiment of the apparatus according to the invention. Basically the apparatus consists of three replaceable units, the receptor 2, the hand unit 1 and the docking station 3. Replacement of the receptor 2 from the hand unit 1 is motivated for hygienic reasons and the requirement of protection against infection. The receptor 2 may easily be contaminated at its position close to the mouth and nose of the test person, and should therefore be exchanged or cleaned before sampling from another test person. Replacement of the hand unit 1 with respect to the docking station 3 is motivated by the fact that the hand unit should be rapidly taken from sleeping state without time consuming function tests. The requirement of a reliable voltage supply is another reason.

The receptor 2 is typically shaped like a cup, mug, funnel or scoop, with a larger inlet opening 24 which is larger than the mouth and nose openings of the test person, and when directed towards them at sampling will effectively receive the expiratory air flow which typically forms a jet. Inspiratory air flow, on the other hand, can take more curved paths.

The shape of the receptor 2 defines an inner volume which may be 10-100 ml, depending on the actual application. There is also a smaller inner opening connecting to the hand unit 1, and its measuring cell 4. Preferably, there is also an element 31 for separation of liquid drops and particles. Furthermore, the receptor 2 advantageously includes a mechanical support element for fixation to a body part, e g the nose and chin tips. It may also include means for connecting to a face mask or other aids for assisted breathing. The details of these implementations are not included in FIG. 2.

The inlet opening 24 of the receptor 2 has a cross section area larger than the corresponding areas of nose and mouth openings of a typical test person. The jet-like air stream from the mouth or nose of the test person with good margin will be received at the inner wall of the receptor 2 essentially without loss, and without requiring an accurate position control. Incident air flow is shown in FIG. 2 as three arrows pointing in the right direction. When the jet hits the inner wall of the receptor 2 it will be distributed into one branch which via the inner opening of the receptor 2 passes through the measuring cell and another, expanding branch that first moves from the centre and thereafter gives rise to a reverse flow compared to the incident one. The flow reflection or reflow emanates from the side wall of the receptor which at least partly prevents further radial expansion. The reflow is the origin of the earlier mentioned, concentration preserving effect by protecting the breath sample from being mixed with ambient air.

The hand unit 1 includes a measuring cell 4 designed for IR spectroscopic analysis of gas within the inner volume of the measuring cell. From the IR source 10 electromagnetic radiation is emitted at a relatively broad wavelength range, typically 2-6 μm, by black body radiation. Preferably the IR source is modulated at 2 Hz repetition frequency or higher in order to avoid baseline drift of the system. The measuring cell 4 is enclosed by molded walls 6 having highly reflecting surfaces of gold or aluminum with a reflection coefficient of 0.98 or higher. By multiple reflections against focusing surfaces, the demands on large aperture and long optical path may be combined with small physical dimensions of the measuring cell 4. Typical outer dimensions are 40×50×5 mm, with an inner volume of 5 ml.

The IR detectors 8, 9 are located at suitable positions in order to receive radiation from the IR source 10. The detector 8 is preferably positioned with a shorter optical path to the IR source 10, and adapted to the absorption band of CO₂ at 4.2-4.3 μm, alternatively water vapor at 2,5-2,8 μm. In front of the detector is an interference filter 13 of band pass type, adapted to precisely this wavelength range. Correspondingly, the detector 9 is adapted to detection of organics substances, e g ethyl alcohol, at the wavelength range 3.3-3.5 μm, and is therefore positioned with a longer optical path using an interference filter 14. With an optical path of 20 cm, a resolution of 0.02 mg/l ethyl alcohol is obtained. Higher resolution may be obtained by increasing the optical path, alternatively using a detector 9, and amplifier 12 with lower noise.

In a preferred embodiment of the invention it is possible to stop the inflow of air into the measuring cell at sampling and zeroing. At sampling this can take place when the CO₂ concentration has reached a certain threshold value. Absence of flow will minimize disturbances and noise caused by air movement within the measuring cell 4. Thereby maximum resolution can be obtained for the determination of volatile organic substances.

When there is a demand for identification of organic substances, the detector 9 may include a multiple set of filters 14 in the wavelength range 3.0 to3.6 μm. By analyzing the relative fractions of the signals within this range, it is possible to identify individual substances, due to the fact that their molecular structure gives rise to individually different fine structure of the absorption peak.

The filters 13, 14 may in an alternative embodiment be replaced by other dispersive elements for multi variable analysis, e g diffraction gratings, which may be advantageous due to their low fabrication cost.

The transport of the breath sample via the receptor 2 through the measuring cell 4 is preferably performed actively by a pump 16, via tubing 25, 26, 27 to the outlet opening 28 to ambient air. The pump 16 is advantageously closed when not activated, e g by embedded non-return valves. The pump 16 may be peristaltic or using membrane, centrifuge or cog wheels. Typically, the volume flow is 1-10 ml per second. Air flow to the measuring cell may preferably be closed at all times except during zeroing and sampling in order to protect the measuring cell 4 from environmental influence when the apparatus is in a sleeping or storage state.

The measuring cell preferably includes a heating arrangement 7 by which the reflecting surfaces 5 are heated to body temperature or above. The purpose of the arrangement 7 is to avoid condensation of water droplets on the reflecting surfaces. Preferably, a pressure transducer 20 is used for monitoring the pressure drop resulting from activation of the pump. Thus it is possible to detect whether or not the receptor 2 is correctly connected. Alternatively, a dedicated sensor arrangement 21, 22 is used for identification and detection of presence of the receptor 2.

The signals from the IR-detectors 8, 9, the pressure transducer 20, the sensor 21 and the start button 23 are taken via amplifiers 11, 12 to a microprocessor 18, in which signal processing takes place according to a preprogrammed sequence governed by the pattern described in the flow chart of FIG. 1. The microprocessor 18 has capacity to execute analog to digital conversion and to emit control signals to the IR source 10, the heating arrangement 7, the pump 16 via buffer stage 19, and the display 15.

The display 15 presents information about the results of the analysis, and the various states of the system by graphics, color or alphanumerical characters, preferably visible also at unfavorable illumination conditions, and observation angles.

The hand unit 1 is powered by a rechargeable battery 17, and its condition is symbolically shown on the display 15. When the hand unit 1 is connected to the docking station 3, the battery 17 is automatically connected to a power unit 29 which via mains or other sources will supply it with necessary energy to manage required number of breath analyses until next docking. At docking, connection between the microprocessor 18 and another microprocessor 30 in the docking station is established. Then more extensive function control is executed along with data communication providing backup of information stored in the local memory of the hand unit.

It should be noted that the various components and blocks of FIG. 2 are not drawn according to their physical size. The hand unit 1 preferably has a volume of 500 ml or less to meet the demands on simple use. The hand unit 1 is preferably designed for single handed use made possible by the fact that the start button 23 only is required for operating the unit. Presetting, programming, calibration and other adjustments are preferably performed when the unit is in the docking condition, and connected to a computer.

FIG. 3 schematically shows a preferred embodiment of the receptor according to the invention. In this embodiment the receptor is coaxially divided into one inner inlet channel and one outer outlet channel with reverse flow direction.

From the mouth or nose opening 40 a flow of expiratory air is drawn as three arrows pointing to the right direction. The receptor 41 has been positioned adjacent to, and at a relatively close distance from the nose/mouth opening 40, without necessarily touching it or any other body part. The receptor 41 has an outer wall 42 which together with the opening towards the mouth/nose opening 40 defines a certain volume, the size of which does not exceed the desired sample volume. Furthermore, the receptor 41 includes an inner wall 43 which like the outer wall 42 opens itself towards the mouth/nose opening 40. The opening of the inner wall 43 preferably has an area which is entirely enclosed by the opening of the outer wall 42. The walls 42 and 43 are fixed against each other with pins or correspondingly, not shown in the figure, and which have minimum flow resistance. Seen from the mouth/nose opening 40 the receptor 41 has two concentric openings. That the walls 42 and 43 have been drawn with circular cross section should be seen as examples of design without preference. Other shapes are possible and may be advantageous.

The opening 47 of the receptor 41 is preferably designed such that it does not by accidence block the mouth/nose opening 40 and thereby preventing the test person from breathing. The walls 42 and 43 preferably are equipped with one or several side holes 48 for that purpose, having a total cross section area of at least 1 cm². Along the symmetry axis of the receptor 41, there is a receiving tube 44 with connection 45 to the measuring cell of the apparatus.

Respiratory air from the mouth/nose opening 40 is incident through the opening 47 of the inner wall 43 and is then distributed into one branch through the receiving tube 44 to the measuring cell, and another branch following the curvature of the outer wall 42, the shape of which leads the air flow backwards in the opposite direction to the incident air flow. The inner wall 43 in combination with the curvature of the outer wall 42 prevents eddy formation and turbulence. The opening 47 and the intermediate air layer 46 define an inner and outer flow channel with opposite flow directions.

The method and apparatus according to the invention can be varied in many ways within the framework of the claims described below.

1. Method for breath analysis including
sampling by air intake to a measuring cell (4) through the inlet opening (24) of a receptor (2; 41) having concentration preserving effect, located adjacent to the mouth and nose opening of a test person in order to receive flow of expired air
whereby said flow is distributed by the shape of said receptor into one branch leading to said measuring cell, and into another branch flowing back to said inlet thereby protecting incident flow from being mixed with ambient air, whereby said concentration preserving effect is obtained, and
determination by means of said measuring cell the concentration of a volatile organic substance, e g ethyl alcohol, and a physiological reference substance, e g carbon dioxide.

2. Method according to claim 1 characterized by manual starting command for breath sampling from operator, duration of air intake being controlled by sensor signal from said measuring cell (4), result presentation taking place during or immediately after finished sampling, and air flow at sampling and zeroing being stopped before analysis of organic substance.

3. Method according to claim 1 characterized in that sampling comprises a volume and air flow not exceeding 50 ml and 10 ml per second, respectively, at insignificant flow resistance, sampling is essentially contactless, and that air intake is controlled by pumping element (16).

4. Method according to claim 1 characterized in that zeroing and function test is automatically executed and includes control with respect to absolute value of time variation of measuring variable, and presence of receptor (2; 41).

5. Method according to claim 1 characterized by said determination is performed by IR spectroscopic analysis, and includes the presence of other volatile organic substance than ethyl alcohol within said breath sample.

6. Method according to claim 1 characterized in that the timing of sampling is controlled by starting command from operator, and the duration of air intake is controlled by sensor signal from the measuring cell.

7. Breath analyzer comprising measuring cell (4), and receptor (2, 41) connected to said measuring cell including an inlet opening (24) which during sampling is directed towards the mouth and nose of a test person in order to receive expired air, and a connector (45) to the measuring cell characterized in that the receptor (2, 41) has the shape of a cup, funnel or scoop with a geometry such that the expiratory air flow received at said inlet opening (24) is partly fed to said measuring cell and partly reflowing back to said inlet opening (24).

8. Breath analyzer according to claim 7 characterized in that said receptor (2; 41) is exchangeable and includes element for separation of liquid drops or particles from said breath sample, and mechanical support element for fixation to a body part, e g the nose and chin tips.

9. Breath analyzer according to claim 7 characterized in that said measuring cell (4) includes source, reflector, dispersive element and detector for multivariable IR spectroscopic analysis.

10. Breath analyzer according to claim 7 characterized in that it includes single-hand operated hand unit 1, exchangeable receptor (2; 41) and docking station 3 for placement of said hand unit (1) when inactivated.
